(19) United States
(12) Patent Application Publication
(10) Pub. No.: US 2003/0059839 A1
(43) Pub. Date: Mar. 27, 2003

(54) Method for detecting pathogens using immunoassays

(76) Inventors: Richard J. Obiso, Frederick, MD (US); Charles C. Young, Mt. Airy, MD (US); Eddie P. Jeffries, Sterling, VA (US)

Correspondence Address: NIXON & VANDERHYE P.C., 8th Floor, 1100 North Glebe Road, Arlington, VA, 22201, US

(21) Appl. No.: 10/151,295

(22) Filed: May 21, 2002

Related U.S. Application Data
(60) Provisional application No. 60/292,777, filed on May 22, 2001.

(51) Int. Cl. G01N 33/53

(52) U.S. Cl. 435/7.1

(57) ABSTRACT

A method is described for the detection, either qualitatively or quantitatively, of gastrointestinal microorganisms in samples, preferably fecal samples, using immunoassays, preferably electrochemiluminescence (ECL) immunoassays. The methods involve detection without the conventional processing steps typically required for such detection (e.g., lysing, enrichment, separation, or purification). The method comprises dilution of the sample thought to contain a specific microorganism, inactivation (e.g., heat inactivation) of the diluted sample, removal of solids from the inactivated diluted sample, and detection of the pathogen in the inactivated diluted sample, by conducting an electrochemiluminescence assay for a bound complex of antibody and antigenic derivative, thereby detecting and/or presumptively identifying the microorganism in the sample.

The present application claims benefit of Provisional Application No. 60/292,777, filed May 22, 2001, the entire contents of which is hereby incorporated by reference.

### FIELD OF THE INVENTION

This invention relates to methods, reagents and kits for identifying, and/or measuring specific bacterial pathogens from samples in an industrial, environmental, or clinical microbiology setting.

### BACKGROUND OF THE INVENTION

Infectious diarrhea caused by specific intestinal pathogens like Campylobacter, E. Coli, Salmonella, Listeria and Shigella account for hundreds-of-millions of cases each year, worldwide. Foodborne infections are a consequence of the growth of specific pathogenic bacteria in the intestines of humans. Symptoms of infection include diarrhea, nausea, vomiting, and fever. The organisms that cause infection often possess several virulence factors that allow them to attach and multiply in the intestines. These microorganisms can quickly grow to large numbers (e.g., as high as 10⁹ bacteria/gram of feces). Several of these bacteria also produce toxins and other factors that can damage cells and cause an outpouring of fluid into the intestines. Death of a patient can occur from dehydration, from systemic invasion of the bacteria through the intestinal wall, or from specific damage by toxins.

Many different microorganisms have been associated with diarrhea in humans. Many laboratories routinely screen for Salmonella, Shigella, Listeria and Campylobacter, typically in fecal samples from symptomatic patients. However, biotechnological advances in the microbiology laboratory are still in early development, so the microbiologist is left using culture methods, which can take days to complete and can cause delays in treatment. Detection of most gastrointestinal pathogens in fecal samples is based on the isolation of the pathogen using selective agar plates, followed by biochemical identification and possible serological classification. These procedures may take as long as 4 days to complete.

In recent years, screening tests that are not based on culture methods have become available for eliminating negative specimens. These tests, however, tend to be significantly less sensitive than culture-based methods and/or also require time consuming or complex manipulations. The most widely used screening methods for the detection of specific bacteria are immunoassays based on the use of specific antibodies directed against the pathogen of interest. Immunoassay formats that have been used to detect specific bacteria, include: ELISA (enzyme linked immuno-sorbent) assays, lateral flow membrane assays (e.g., strip tests and dip sticks), and latex-bead agglutination assays. Commercial immunoassay tests for gastrointestinal pathogens have sensitivities (typically 10⁶-10⁸ cells per sample) that are, generally, considerably worse than culture methods; these tests, therefore, may miss infected patients that would otherwise have been identified by culture methods. These immunoassay tests also, typically, require cumbersome and time consuming sample preparation steps including i) enrichment and/or concentration of the bacteria to levels that are detectable by the assay (e.g., enrichment by growth of bacteria in enrichment media or concentration by immunoprecipitation, collection on filtration membranes, centrifugation, etc.); ii) purification of the bacteria to remove interfering materials in complex sample matrices (e.g., by immunoprecipitation, collection on filtration membranes, centrifugation, etc.); or iii) lysing of the bacteria to expose internal antigens that are present in high copy number (e.g., by chemical means such as through detergents, organic solvents, strong oxidants, etc. or physical means such as sonication or homogenization). Conventional immunoassay tests may also suffer from slow binding kinetics and a requirement for one, or often multiple, wash steps.

Immunoassays for pathogens in fecal samples are especially problematic. The large intestines of most mammalian species contain the most complex and concentrated bacterial environment in existence. Typically, there are more bacteria found in 1 gram of fecal matter than the number of people that have ever existed on the Earth. Detection of a small population of a specific pathogenic bacteria in this large population of bacteria requires exceptional sensitivity and selectivity. The contents of the mammalian intestine are literally bathed in proteases, nucleases, glycosidases and cell-lytic enzymes. In addition, there are large amounts of particulate materials and high concentrations of toxic chemicals like bile acids, mucous-like polysaccharides, and oxygen radicals. Assays on fecal samples must, therefore, be highly robust and tolerant of these potential interferents. With respect to assays requiring bacterial enrichment, the enrichment of bacterial in fecal samples is controversial because of the presence of such a rich milieu (there may be as high as 10¹¹ bacteria/gram of feces) of competing normal flora bacteria; the growth of the bacteria of interest may be highly variable and dependent on the nature and amount of the competing species. Furthermore, growth of fecal bacteria on selective media can take as long as 48 to 72 h to complete. As a result, there is a need for a fast, sensitive and reliable method for the presumptive identification of bacteria in feces without the need for enrichment or plating methods.

Recently, methods have been developed to detect fecal pathogens by detecting their genetic material. These methods may employ nucleic acid hybridization reactions, PCR (polymerase chain reaction), RFLP (restriction fragment length polymorphism) analysis, or other molecular biology technologies. As in the case of immunoassays, the use of molecular techniques for detecting pathogens in feces may be complicated by interferents in the complex sample matrix. For example, the detection of specific pathogenic bacteria directly in human fecal specimens by PCR or RFLP analysis requires removal of PCR-inhibitory substances. Several investigators have looked at various filters, centrifugation, cell washing, and other methods that could remove polysaccharides, which are major PCR inhibitors. Currently, a combination of several steps is required to remove PCR inhibitory polysaccharides. These steps are time-consuming, variable, and may yield a slight risk of pathogen exposure to the user. Furthermore, detection of bacteria in fecal samples by any number of molecular methods (PCR, RFLP, DNA-hybridization, 16S ribosomal-RNA detection) may require additional steps to lyse cells, extract nucleic acids, purify nucleic acids and amplify nucleic acids for analysis. Thus, these methods are technically complex and require hours of sample preparation.

A. Campylobacter Species

Campylobacter are a group of several microaerophilic species that are slender, curved, and motile Gram-negative rods. Even though Campylobacter has been known as a foodborne pathogen since the early 1970's, it is classified as a new and emerging pathogen, and as a result of its anonymity, few rapid tests for Campylobacter are available. There are approximately 4 million cases of Campylobacter in the United States annually with only a 1-5% reporting rate. The disease is also associated with an autoimmune disease called Gullain-Barre syndrome. Campylobacter infections are characterized by diarrhea, which may be watery or sticky and may contain blood and fecal leukocytes. Other symptoms often present are fever, abdominal pain, nausea, headache, and muscle pain. The illness occurs after a 2-5 day incubation period and can persist for 7-10 days.

B. Salmonella Species

Salmonella is a rod-shaped, non-sporeforming Gram-negative bacteria that is found in many widespread environments, including: animals, water, soil, insects, plants, and other surfaces. The genus, Salmonella, comprises 4 major groups, of which include approximately 3200 different serotypes. Salmonella causes acute infectious diarrhea in a number of mammalian species, including humans. The infective dose is approximately 15-1000 cells and onset until symptoms becomes apparent in approximately 6-48 hours. Symptoms include diarrhea, abdominal-pain, fever, vomiting, and dehydration. It is estimated that there are at least 2 to 4 million cases of salmonellosis in the United States annually. The mortality rate for salmonellosis is as high as 1% and 10% for typhoid fever (a specific type of Salmonella infection caused by S. typhi). Outbreaks of the disease have been known to occur on a regular basis, with approximately 150 major outbreaks of Salmonella occurring in the United States to date.

There are numerous immuno-based test kits for Salmonella (ELISA and Lateral Flow (e.g., Dipstick) methods) on the market, but none of them are more sensitive than the traditional microbiological plating methods and they can take as long as 48-50 hours from sampling to obtain a result. PCR-based methods also exist; however, these require extensive sample processing and do not differentiate between live or dead organisms.

C. E. coli Serotypes

E. coli O157 infection often causes severe bloody diarrhea and abdominal cramps, sometimes the infection causes non-bloody diarrhea or no symptoms. Usually little or no fever is present, and the illness resolves in 5 to 10 days. E. coli O157 is one of hundreds of strains of the bacterium Escherichia coli. Although most strains are harmless and live in the intestines of healthy humans and animals, this strain produces a powerful toxin and can cause severe illness. E. coli O157 was first recognized as a cause of illness in 1982 during an outbreak of severe bloody diarrhea; the outbreak was traced to contaminated hamburgers. Since then, most infections have come from eating undercooked ground beef. An estimated 73,000 cases of infection and 61 deaths occur in the United States each year. Infection often leads to bloody diarrhea, and occasionally to kidney failure. Infection with E. coli O157 is typically diagnosed by detecting the bacterium in the stool.

### SUMMARY OF THE INVENTION

The present invention relates to improved methods, preferably electrochemiluminescence-based methods, for identifying, measuring and/or detecting the presence of a pathogen in a sample. Preferably, the methods are more sensitive, more accurate, faster, less complicated or require fewer steps than conventional methods. The present invention also relates to improved methods for identifying, measuring and/or detecting the presence of a pathogen in a complex matrix such as a stool sample. Preferably, specific bacterial pathogens are measured directly from a sample (e.g., stool) without enrichment, lysing, concentration, and/or purification of the bacterial pathogen. Moreover, the methods of this invention reduce or eliminate cumbersome and time-consuming washing and/or sample handling steps.

The limited sample preparation, the simple assay steps, and the use of electrochemiluminescent labels according to this invention allows for the rapid and direct detection of foodborne pathogens in samples such as fecal specimens. The present method may be used for rapidly identifying pathogens, preferably specific pathogens that cause disease. The use of the method to detect pathogens in clinical samples, in general, and stool samples, in particular, should lead to faster diagnosis and treatment, and ultimately, better healthcare. The methods of the invention may also be applied to the rapid detection of pathogens in environmental samples, wastewater, drinking water, foods and beverages.

The invention also relates to kits comprising, in one or more containers, one or more assay components selected from the group consisting of i) one or more assay reagents used in a method of the invention; ii) one or more calibrators or controls for use in a method of the invention; and iii) one or more devices, e.g., filters, centrifuge tubes, etc. for use in a method of the invention.

### BRIEF DESCRIPTION OF THE DRAWINGS

The present invention is described with reference to the accompanying drawings, wherein:

FIG. 1 is a graphical representation of a matrix analysis of the sensitivity of a Campylobacter test according to one embodiment of the invention wherein the first horizontal axis represents pathogen concentration (CFU/mL), the second horizontal axis represents sample number and the vertical axis represents signal/background ratio.

FIG. 2 is a graphical representation of a matrix analysis of the sensitivity of an E. coli O157 test according to one embodiment of the invention wherein the first horizontal axis represents pathogen concentration (CFU/mL), the second horizontal axis represents sample number and the vertical axis represents signal/background ratio.

FIG. 3 is a graphical representation of a matrix analysis of the sensitivity of a Salmonella test according to one embodiment of the invention wherein the first horizontal axis represents pathogen concentration (CFU/mL), the second horizontal axis represents sample number and the vertical axis represents signal/background ratio.

### DETAILED DESCRIPTION OF THE INVENTION

The present invention relates to improved methods for detecting pathogens in samples. The methods provide useful, rapid results in extremely complicated samples such as stool samples without the need for enrichment, concentration, or purification of the pathogen.

In one embodiment of the invention, the method comprises the step of conducting an immunoassay, preferably a sandwich immunoassay, for a pathogen in a sample, preferably a stool sample. Preferably, the immunoassay is an electrochemiluminescence-based immunoassay. Preferably, the method excludes one or more sample pretreatment steps selected from growth, lysis, concentration and purification of the pathogenic bacteria. Preferred methods of the invention can detect (surprisingly, even in complex samples such as stool) less than 10⁶ bacterial cells, more preferably less than 10⁵ bacterial cells, even more preferably less than 10⁴ bacterial cells, even more preferably less than 10³ bacterial cells and most preferably less than 10² bacterial cells, without enrichment of the sample. Optionally, detection of less than 10², more preferably less than 10 and most preferably 1-10 cells can be achieved by including an enrichment step.

One embodiment of the invention relates to a method for detecting a bacterial pathogen in a fecal sample comprising:

(a) forming a composition containing said fecal sample and an assay-performance-substance linked to an electrochemiluminescent compound and containing at least one component selected from the group consisting of:

(1) added bacterial pathogen or added analogue of said bacterial pathogen,

(2) a binding partner of said bacterial pathogen or a binding partner of said analogue; and

(3) a component capable of binding with (1) or (2); and

(b) inducing electrochemiluminescence from said electrochemiluminescent compound; and

(c) detecting emitted luminescence.

Preferably, the method does not include a sample enrichment step.

Preferably, the method consists essentially of steps (a)-(c).

Another embodiment of the invention relates to an electrochemiluminescence based immunoassay for detecting a pathogen in a sample wherein said assay method does not comprise an enrichment step.

In preferred embodiments of the invention, the assay method includes pretreatment steps that allow for solubilizing of a component of the pathogen, rapid removal of particulate matter from the sample and/or inactivation of the organism, e.g., one or more of the steps of:

a. diluting a sample, preferably a fecal sample, in a buffer to dilute and/or suspend the particulates;

b. solubilizing a component of the pathogen of interest in the sample, preferably by the addition of detergent;

c. inactivating, preferably heat inactivating, the sample to destroy the potential for contamination; and/or

d. separating large solids from the bacterial sample, preferably by decanting, filtering or centrifuging.

Preferably, the sandwich immunoassay of the invention comprises

a. forming a composition comprising the sample (preferably, a pretreated sample as described above), a first antibody linked to a label (preferably, an ECL label) and a second antibody and

b. forming a sandwich immuno-complex comprising the first antibody, the second antibody and the pathogen of interest or a component thereof; and

c. measuring the formation of the sandwich immuno-complex (preferably by inducing said ECL label in the sandwich immuno-complex to emit ECL and measuring the emitted ECL) so as to measure or identify the pathogen of interest in the sample.

In certain preferred embodiments of the invention, the second antibody is linked to a solid phase support so that the sandwich complex is formed on the solid phase support and the detecting of the sandwich immuno-complex comprises measuring the amount of label on the solid phase. Particles, preferably magnetic particles, are an especially advantageous solid phase support. Electrochemiluminescence-based methods of the invention may further comprise the steps of collecting the particles on an electrode (preferably via the application of magnetic field), inducing electrochemiluminescence and measuring emitted electrochemiluminescence.

Optionally, the second antibody and the solid phase support (e.g., magnetic particles) may be provided as separate assay components that are linked as part of the assay procedure, e.g., through a biospecific linkage. For example, the solid phase support (e.g., a magnetic particle) may be coated with Protein A, Protein G, Protein L or a secondary antibody (e.g., a goat anti-mouse antibody, etc.) that binds the second antibody via a bio-specific interaction. Alternatively, the second antibody may be labeled with a capture moiety (e.g., biotin, a hapten, an epitope tag etc.) and the solid phase support (e.g., a magnetic particle) coated with a capture reagent (e.g., avidin, streptavidin, anti-hapten antibody, anti-epitope tag antibody, etc.) that binds the capture moiety. Accordingly, the assay method may further comprise the step of capturing the second antibody on the solid phase support via a bio-specific interaction. This capture step may occur before, during or after the formation of the sandwich complex. In especially preferred embodiments of the invention, the second antibody is labeled with biotin and the solid phase support is magnetic particles coated with streptavidin.

In additional embodiments of the invention, a competitive assay format is used comprising the steps of

a. forming a composition comprising the sample (preferably, a pretreated sample as described above), an antibody and a competitor, wherein the competitor is a species that competes with the pathogen of interest, or a component thereof, for binding to the antibody and wherein said antibody or said competitor is linked to a label (preferably, an ECL label);

b. forming a complex comprising said antibody and said competitor; and

c. measuring the formation of said complex so as to measure or identify the pathogen of interest in the sample.

One of the antibody and competitor, preferably the one not linked to a label, may be linked or capable of being linked (via a bio-specific interaction as described above, e.g., using a secondary antibody or via a capture moiety-capture reagent interaction) to a solid phase support. By analogy to the description given above for sandwich assays, the assay method may further comprise the step of capturing one of the antibody or competitor on a solid phase support via a bio-specific interaction. In an especially preferred embodiment, one of the antibody or competitor is linked to biotin and the solid phase support is magnetic particles coated with avidin or streptavidin.

It should be noted that while the methods described above use antibodies as binding reagents for binding the analyte and/or for capturing antibodies on solid phase supports, any or all of the antibodies may be replaced with any other binding reagents having suitable binding affinity and selectivity, e.g., binding proteins other than antibodies (e.g, receptors that bind components of an organism of interest), nucleic acids selected by the SELEX process, etc. It should also be noted that the methods are also applicable to the measurement and identification of non-pathogenic organisms as well as pathogens.

In one embodiment of the invention, the present method overcomes the deficiencies of prior art methods by providing a rapid method for detection and/or presumptive identification of specific intestinal pathogens comprising the steps of

a. diluting a sample, preferably a fecal sample, in a buffer to dilute and/or suspend the particulates;

b. inactivating, preferably heat inactivating, the sample to destroy the potential for contamination;

c. separating large solids from the bacterial sample, preferably by decanting, filtering or centrifuging;

d. adding an assay-performance-substance (preferably, an antibody) to the filtered sample, the substance being linked to a label, preferably an electrochemiluminescent compound, and being capable of specifically binding with a target microorganism of interest in the sample or a product of the target;

e. inducing a signal, preferably electrochemiluminescence;

f. and detecting or quantitating emitted signal.

Optionally, steps (a), (b) and/or (c) may be omitted. Preferably, the method also comprises incubating the sample to form a complex containing collectible particles and the electrochemiluminescent compound. More preferably, the method further comprises collecting the complex in a zone where electrochemiluminescence can be induced to occur.

One advantage of the preferred embodiments of the present invention is that the methods are truly rapid and minimize actual labor input to approximately 15-30 minutes of hands-on-time. The total assay time from start to finish can occur in less than 1.5 hours.

Another advantage of certain preferred embodiments of the invention is the use of direct detection (e.g., they do not require growth enrichment of bacteria). The problem with enrichment of bacteria is that the nutrient and metabolite concentrations comprise a microenvironment and are continually changing. The heterogeneity of samples, especially samples that contain several different types of microorganisms, cannot be accurately replicated and may not give an accurate representation of the “true” presence or concentrations of specific microorganisms. Therefore, direct detection, especially in fecal samples, is probably the best avenue to accurately determine the presence of a particular microorganism. In addition, direct detection avoids potential problems such as growth competition with other microorganisms, stressors, nutrient and growth issues, which may interfere with the assay.

The invention preferably provides a rapid method for the direct detection and presumptive identification of specific bacterial pathogens in fecal samples. The method can be extended to the detection and presumptive identification of specific bacterial pathogens in food, water, and environmental samples.

Additional applications of this invention will become apparent to one of ordinary skill in the art from the disclosure of the invention, below.

There are currently a number of commercially available instruments that utilize electrochemiluminescence (ECL) for analytical measurements. ECL is the emission of light caused by reactions of electrically stimulated species (see, e.g., Leland and Powell, 1990 J. Electrochem. Soc. 137(10) 3127-3131). Species that can be induced to emit ECL are termed ECL labels or ECL-active species and are also referred to herein as ECL TAGs. Commonly used ECL labels include: i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Preferred electrochemiluminescent labels for use with the methods of the invention are polypyridyl (e.g., bipyridine and phenanthroline-containing) complexes of Ru or Os. Especially preferred electrochemiluminescent labels are labels that comprise Ru(bpy)₃ or derivatives thereof. Species other than ECL labels that participate in a reaction that causes ECL are referred to herein as ECL coreactants. Commonly used coreactants include tertiary amines (e.g., see U.S. Pat. No. 5,846,485, herein incorporated by reference), oxalate, and persulfate for ECL from RuBpy and hydrogen peroxide for ECL from luminol (see, e.g., U.S. Pat. No. 5,240,863, herein incorporated by reference). Preferred coreactants for use with the electrochemiluminescent assays of the invention include tertiary amines, more preferably trialkylamines, most preferably tripropylamine. The light generated by ECL labels can be used as a reporter signal in diagnostic procedures (Bard et al., U.S. Pat. No. 5,238,808, herein incorporated by reference). See also, Massey et al., U.S. Pat. No. 6,316,607, herein incorporated by reference. For instance, an ECL label can be covalently coupled to a binding agent such as an antibody or nucleic acid probe; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants, herein incorporated by reference). For more background on ECL, ECL labels, ECL assays and instrumentation for conducting ECL assays see U.S. Pat. Nos. 5,093,268; 5,147,806; 5,324,457; 5,591,581; 5,597,910; 5,641,623; 5,643,713; 5,679,519; 5,705,402; 5,846,485; 5,866,434; 5,786,141; 5,731,147; 6,066,448; 5,776,672; 5,308,754; 5,240,863 and 5,589,136 and Published PCT Nos. WO99/63347; WO00/03233; WO99/58962; WO99/32662; WO99/14599; WO98/12539; WO97/36931 and WO98/57154, each of which are herein incorporated by reference.

One embodiment of the invention relates to a method for detecting a bacterial pathogen, preferably a specific genus of pathogen, more preferably, two or more specific pathogens, in a sample comprising:

(a) diluting the sample to form a diluted sample;

(b) forming a slurried sample from the diluted sample;

(c) optionally, inactivating the slurried sample to form an inactivated slurried sample;

(d) forming a composition containing:

(i) the inactivated slurried sample;

(ii) an assay-performance-substance linked to an electrochemiluminescent compound and containing at least one component selected from the group consisting of

(1) added bacterial pathogen or added analogue of the bacterial pathogen;

(2) a binding partner of the bacterial pathogen or a binding partner of the analogue; and

(3) a component capable of binding with (1) or (2); and

(iii) a plurality of particles capable of specifically binding with the bacterial pathogen, the assay-performance-substance or combinations thereof;

(e) incubating the composition to form a complex containing the particles and the electrochemiluminescent compound;

(f) inducing electrochemiluminescence from the electrochemiluminescent compound; and

(g) detecting emitted luminescence.

Preferably, the method consists essentially of steps (a) through (g), more preferably the method consists of steps (a) through (g).

According to one preferred embodiment, steps (a) through (g) are performed in less than 6 hours, preferably less than 5 hours, more preferably less than 4 hours, even more preferably less than 3 hours, even more preferably less than 2 hours, even more preferably less than 1.5 hours and most preferred less than 1 hour.

Another embodiment of the invention relates to method for detecting a bacterial pathogen, preferably one or more specific pathogens, in a sample comprising:

(a) diluting the sample to form a diluted sample;

(b) forming a slurried sample from the diluted sample;

(c) optionally, inactivating the slurried sample to form an inactivated slurried sample;

(d) removing large solids from the inactivated slurried sample to form a supernatant sample;

(e) forming a composition containing:

(i) the supernatant sample;

(ii) an assay-performance-substance linked to an electrochemiluminescent compound and containing at least one component selected from the group consisting of:

(1) added bacterial pathogen or added analogue of the bacterial pathogen;

(2) a binding partner of the bacterial pathogen or a binding partner of the analogue; and

(3) a component capable of binding with (1) or (2); and

(iii) a plurality of inanimate particles capable of specifically binding with the bacterial pathogen and/or the assay-performance-substance;

(f) incubating the composition to form a complex containing the inanimate particles and the electrochemiluminescent compound;

(g) collecting the inanimate particles in a zone where electrochemiluminescence can be induced to occur;

(h) inducing electrochemiluminescence; and

(i) detecting emitted luminescence.

Preferably, the method consists essentially of steps (a) through (i), more preferably the method consists of steps (a) through (i).

According to one preferred embodiment, steps (a) through (i) are performed in less than 6 hours, preferably less than 5 hours, more preferably less than 4 hours, even more preferably less than 3 hours, even more preferably less than 2 hours, even more preferably less than 1.5 hours and most preferred less than 1 hour.

The assay performing substances of the invention are generally either i) binding reagents that bind the analyte of interest or a component thereof; ii) competitors that compete with the analyte of interest or a component thereof in a binding interaction or iii) additional binding reagents that bind to either (i) or (ii). Additional binding reagents refer, e.g., to components of systems for indirectly labeling a binding reagent such as labeled secondary antibodies used to link a label a primary antibody or to a biotin-label conjugate used to label a biotin-labeled antibody via the use of avidin as a cross-linking agent. The binding agents that bind the analyte of interest are, preferably, antibodies although any binding reagents with suitable affinity and specificity may be used. Competitors may be any material that competes with the analyte of interest in a binding reaction but are, generally, labeled analogs of the analyte.

Another embodiment of the present invention provides a direct and rapid method for the detection and/or the presumptive identification of specific bacterial pathogens, including Campylobacter species, E. coli (preferably, E. coli O157), Shigella, Listeria species, and Salmonella species from fecal samples.

Preferably, the pathogen is selected from Campylobacter species, Shigella, Listeria species, and Salmonella species from food or fecal samples, preferably fecal samples.

In a preferred embodiment, the method comprises the steps of:

a. diluting a fecal sample in a buffer to dilute particulates to approximately 10% weight per volume;

b. vortexing the fecal sample until it becomes a fecal slurry;

c. heat inactivating the sample to destroy the potential for contamination;

d. filtering the sample to remove solids through a filter of approximately 200 um;

e. adding to the filtered sample (i) an assay-performance-substance linked to an electrochemiluminescent compound and containing at least one component selected from the group consisting of:

(1) added pathogen or added analogue of said pathogen.

(2) a binding partner of said pathogen or a binding partner of said analogue; and

(3) a component capable of binding with (1) or (2) and

(ii) a plurality of inanimate particles (e.g., preferably magnetic particles) capable of specifically binding with the pathogen and/or the assay-performance-substance;

f. incubating the sample to form a complex containing the particles and electrochemiluminescent compound;

g. collecting the sample in a zone where electrochemiluminescence can be induced to occur;

h. inducing electrochemiluminescence; and

i. detecting or quantitating emitted luminescence.

Preferably, the sample is an unenriched sample and/or a “direct sample” that has not been subjected to lysing, enrichment, separation or purification.

According to one preferred embodiment, the sample is selected from the group consisting of a food sample, a fecal sample (preferably a human sample), or a water sample. According to a particularly preferred embodiment, the sample is a fecal sample. Fecal samples can be frozen or fresh so they can be instantly diluted and tested. According to one preferred embodiment, the sample is a fecal sample less than 2 hours old, preferably less then 1 hour old, more preferably less than 45 minutes old, even more preferably less than 30 minutes old and most preferred less than 15 minutes old.

According to another embodiment, the sample is a direct fecal sample.

According to yet another embodiment, the sample is a fecal sample and the fecal sample is diluted to at least 10% weight/volume in a buffer such as a phosphate buffered saline.

Preferably, the bacterial pathogen is selected from the group consisting of E. coli, Salmonella species, Shigella, Listeria species, and Campylobacter species, more preferably selected from Salmonella species, Shigella, Listeria species, and Campylobacter species.

Preferred methods of the invention provide sensitivities as low as 100 to 50000 cells when detected without enrichment of the sample, and more preferably provide sensitivities as low as 100 to 5000 cells when detected without enrichment of the sample.

According to another preferred embodiment the bacterial pathogen detected is E. coli O157 and the method provides a detection limit of less than or equal to 1×10⁵ CFU/gram of feces without enrichment of the sample.

According to another embodiment, the bacterial pathogen is Salmonella and the method provides a detection limit of less than or equal to 5×10⁵ CFU/gram of feces without enrichment of the sample.

According to yet another embodiment, the bacterial pathogen is Campylobacter and the method provides a detection limit of less than or equal to 1×10⁴ CFU/gram of feces without enrichment of the sample.

According to yet another embodiment, the pathogen is Listeria and the method provides a detection limit of less than or equal to 5×10⁵ cells per milliliter of sample (preferably a food or feces sample) without enrichment of the sample.

Preferably, the slurried sample is formed by vortexing the diluted sample.

Preferably, samples are heat inactivated to kill pathogenic organisms prior to conducting the measurement steps.

According to one embodiment, the step of inactivating comprises heat inactivation, preferably at an inactivating temperature of at least 70° C., preferably 80° C. for between 5 minutes and one hour, more preferably between 10 minutes and 30 minutes and most preferred for approximately 15 minutes.

The solids may be removed from the sample by filtering, centrifugation or the like. Preferably, the solids are removed by filtering, more preferably by filtering through a 200 um filter.

Using the present method, the assay may be performed rapidly. Preferably, the method is performed in less than 5 hours, more preferably less than 4 hours, even more preferably less than 3 hours and most preferred less than 2 hours. According to one particularly preferred embodiment of the invention, the assay is performed in less than about 1.5 hours, preferably less than 1 hour, more preferably less than 45 minutes, even more preferably less than 30 minutes, even more preferably less than 15 minutes, even more preferably less than 10 minutes and most preferred about 5 minutes.

Another aspect of the invention relates to the application of the above-described detection methods to samples requiring an enrichment step. Enrichment is typically necessary when the detection of 1-10 cells is required from a sample. For example, if the detection of 1-10 cells is necessary, the sample may require an enrichment step to facilitate the detection of the pathogen. Using the present invention, the time periods for conventional enrichment procedures can be reduced substantially.

According to the invention, selective enrichment of bacterial pathogens can be accomplished with many types of commercially available liquid and/or solid growth medias. I Applicants have discovered conditions which are particularly advantageous for the selective growth of certain pathogens in samples (described below). Such enrichment is performed using appropriate parameters including appropriate incubation temperatures, oxygen concentrations, nutrient concentrations, salt concentrations and pH conditions. Appropriate parameters include those resulting in the target pathogen being enriched, preferably selectively enriched relative to other bacteria present in the sample. The use of selective agents (antibiotics and/or growth factors) can also be used to more specifically select for certain bacterial pathogens.

One embodiment of the invention includes the enrichment of a sample containing Salmonella species. For example, a protocol for enrichment of Salmonella species from food includes inoculating 25 grams of a food sample into 225 ml of buffered peptone water (Difco/Becton Dickinson) and incubating that solution at 37° C. for 18-24 hours, followed by a 1:100 dilution of that solution into RVS broth (Rappaport Vassiliadis Soyabroth, Becton Dickinson) and incubation at 42° C. for an additional 24 hours. Other selective media capable of enrichment of Salmonella species can also be used such as Brilliant Green broth.

An alternative enrichment protocol for the enrichment of Salmonella includes 18-24 hour enrichment in buffered peptone water (25 g into 225 ml) at 37° C. followed by 6-24 hour enrichment (more preferably less than 12 hours enrichment, even more preferably less than 8 hours and most preferably less than about 6 hours) in RVS broth at about 42° C.

Another embodiment relates to enrichment of a sample containing E. coli. As an example, the protocol for the enrichment of E. coli O157 for a 25 gram sample is incubation with 225 ml of EC broth (Difco/Becton Dickinson) modified with Novobiocin (20 mg/l) for 24 hours at 37-42° C. Alternative methods include incubation in the same media for only 6 hour at 42° C. One preferred embodiment of the invention involves the enrichment of E. coli using a total enrichment time less than 15 hours, preferably less than 10 hours, even more preferably less than about 6 hours.

As another example, the protocol for the enrichment of Campylobacter species is 2 to 6 hours at 37° C. under microaerophilic conditions followed by additional incubation of up to 42 hours at 37° C. to 42° C. in a selective broth (25 gram sample inoculated into 100 ml of broth). Commercially available broths include Bolton (oxiod) or Skirrow Campylobacter broth (BBL/Becton Dickinson) and others. An alternative method includes a 6-hour enrichment at 37° C. in Bolton Broth under microaerophilic conditions (25 g into 100 ml) followed by inoculation of 0.5 ml onto a selective agar media (Campylobacter agar (BBL/Becton Dickinson)) for 18-24 hours under microaerophilic conditions at 37° C. to 42° C.

One preferred embodiment of the invention involves the enrichment of Camplobacter using a total enrichment times less than 30 hours, preferably less than 24 hours.

Another preferred embodiment of the invention relates to a method of enriching Campylobacter using aerobic conditions. As described above, one embodiment of the invention includes growing the Campylobacter under reduced oxygen (microaerophilic). This requires a specialized container that is not gas permeable and a reagent package that converts oxygen to CO₂ inside the container. Surprisingly, in experiments where the pathogen was grown in Bolton Broth under aerobic conditions (and, therefore, conditions that did not require the use of the specialized container) comparable assay results were obtained. In one study using chicken samples from supermarkets, it was noted that if the growth occurred in Bolton Broth under aerobic conditions, the same total number of positive results by plating methods were obtained as when the samples were grown microaerophilically. Also surprising, some of the samples that were grown to levels that could be detected by plating under microaerophilic conditions could not be detected when grown under aerobic conditions and vice versa. Therefore, although the same total number of positives were detected by plating using both growth conditions, each growth condition detected only a subset of the true number of positive samples. Accordingly, in one embodiment of the invention one portion of a sample is enriched under microaerophilic conditions and another portion is enriched under aerobic conditions. Each of the enriched samples is assayed for campylobacter by plating or by immunoassay. By using the two enrichment conditions, it is less likely that the positive samples will be missed.

Another embodiment involves the enrichment of Listeria species using a total enrichment time less than 50 hours, preferably less than about 45 hours, more preferably less than 40 hours. Preferably, the sample is placed in a sterile stomacher-type bag containing enrichment broth (e.g., Becton Dickinson-Difco Listeria Enrichment broth MLEB, catalog #220530) at a ratio of about 1:9 (e.g, about 25 g of food sample with 225 mL of mLEB). Suitable stomacher bags include Spiral Biotech, BagPage®+, Bagfilter®S, Bagfilter®P or equivalent. Preferably, the sample is then incubated at about 30° C. (+/−2° C.) for between 35 and 100 hours, preferably between 35 and 60 hours, preferably between about 40 to 45 hours. The enriched sample is then extracted and tested.

Another aspect of the invention relates to assay methods for measuring Listeria that include using antibodies that and/ordo not target the flagella and/or include growing the Listeria at temperatures greater than 32° C., preferably greater than 34° C., more preferably greater than 35° C., even more preferably greater than 36° C. and most preferably greater than about 37° C. Listeria is typically grown at 30° C. and conventional plating methods and rapid methods also use 30° C. for growth since, for such immunoassays, the antibodies used target flagella. However, Listeria monocytogenes flagella production drops at growth temperatures above around 27-28° C. Although there still is a significant amount of flagella antigen at 30° C., the amount drops at temperatures higher than 30° C. and by 37° C., the flagella antigen greatly reduced. Thus, conventional methods that target flagella are typically confined to using growth temperatures less than or equal to about 30° C. In contrast, according to the invention, the antibodies used target the organism instead of the flagella allowing for the growth temperatures for Listeria to be increased from 30 to 37° C., which provides a shortened enrichment time compared with other tests. Thus, according to the invention, the organisms can be grown at temperatures as high as 37° C. and higher and still work since flagella is not targeted. In fact, Applicants have discovered that many Listeria actually recover and grow better at 37° C. than at 30° C. Since they recover sooner and grow faster, the samples do not have to be enriched as long as other tests. This allows for enrichment times of less than 36 hours, preferably less than 30 hours, more preferably less than 24 hours, as opposed to the typical 40-48 hours. Shorter enrichment times provide faster time to final result. Preferably, this embodiment of the invention is employed in combination with the electrochemiluminescence methods described above.

Another aspect of the invention relates to the use of a detergent or lysing agent to solubilize the pathogen. In food testing, samples are typically heat killed prior to testing. This prevents the spread of the contaminating bacteria throughout the facility and prevents the individuals testing the samples from contracting the pathogen. However, if a heat sensitive reagent is used or if the test measures a heat-sensitive antigen, a heat kill step cannot be used. For example, according to one embodiment of the invention, an antibody which recognizes an antigen on the Listeria that is heat labile is employed. to Therefore, if the Listeria cells are heat killed, the sensitivity of the assays using such antibodies drops significantly. Such antibodies are, generally, made by immunizing animals with antigens from cells that have been killed by treatment with phenol. Applicants, therefore, tried conducting assays using cells that had been killed by treatment with phenol but found that phenol treatment required many hours to kill cells. Surprisingly, the use of a lysing agent (such as B-PER solution from Pierce, a buffer containing a mild non-ionic detergent that was developed for extracting and purifying recombinant proteins expressed in bacteria and has not previously been noted for its advantageous properties in immunoassays of pathogenic organisms) was found to not only kill cells faster and more effectively than phenol but also to give better assay results. Surprisingly, the antibody not only worked in the BPER solution, it worked much better in the BPER solution than it did on live cells. The BPER also killed the pathogen faster than phenol providing complete killing by 10 minutes with a 6-7 log reduction in live bacteria by 5 minutes. Also surprising and unexpected, a 10-fold increase in sensitivity was observed in BPER killed cells over live cells that also had the antigen intact. Surprisingly, the use of a lysing agent or detergent (e.g., BPER) works well not only for the Listeria test but also for E. coli, Campylobacter and Salmonella (although less sensitivity is seen in the Salmonella test compared to heat killing). Preferably, the assay method comprises contacting the sample suspected of containing the pathogen with a detergent (e.g., BPER solution from Pierce) to kill the pathogen and subsequently detecting the presence or amount of pathogen by immunoassay. Preferably, the pathogen is Listeria, E. coli, Campylobacter, Shigella or Salmonella, more preferably Listeria, E. coli, or Campylobacter. Preferably, the method is combined with the electrochemiluminescence assays described above. For example, according to one preferred embodiment, about 0.5 mL of Listeria Sample Diluent is added to each sample tube and 0.1 mL of the sample is added after the Sample Diluent (e.g., a buffered solution comprising a detergent, preferably a non-ionic detergent, most preferably B-PER) and the treated sample is subsequently tested using an ECL assay.

Methods involving enrichment of stool samples are similar to the enrichment methods described above. Typically, 1 gram of feces is inoculated into 9 ml of appropriate media (or 1 part into 10 parts media) and incubated as described above.

The detection protocol for the enriched samples is the same for the non-enriched samples (e.g., inactivation, filtration, detection). Thus, after the sample is enriched it may be subjected to inactivation, filtration, incubation and detection.

Yet another aspect of the invention relates to kits and reagent compositions adapted for use in performing the methods of the present invention. Preferably, the kits and reagent compositions are adapted for use in performing luminescence methods, more preferably electrochemiluminescence methods.

One embodiment of the invention relates to a kit containing, in one or more containers: (a) a first binding partner capable of specifically binding with the pathogen (preferably an antibody, more preferably a monoclonal antibody); (b) a second binding partner capable of specifically binding with the pathogen (preferably an antibody, more preferably a monoclonal antibody); and (c) at least one component selected from: (i) luminescent label, preferably electrochemiluminescent label; (ii) electrochemiluminescent co-reactant, and/or (iii) electrode for inducing electrochemiluminescence.

Another embodiment of the invention relates to a kit for use in a competitive assay containing, in one or more containers: (a) a first reagent comprising added pathogen or an analogue of the pathogen, (b) a second reagent capable of specifically binding with the pathogen (preferably an antibody, more preferably a monoclonal antibody); and (c) at least one component selected from: (i) luminescent label, preferably electrochemiluminescent label; (ii) electrochemiluminescent co-reactant; and/or (iii) electrode for inducing electrochemiluminescence. Preferably, the first reagent is linked to a label (preferably an electrochemiluminescent label) and the second binding partner is attached to a bead, more preferably is attached to a magnetic microparticle having a diameter ranging from 1 to 5 microns. According to another embodiment, the first reagent is linked to the bead and the second binding partner is attached to the label.

Yet another embodiment of the invention relates to a kit containing, in one or more containers: (a) a first binding partner capable of specifically binding with the pathogen (preferably an antibody, more preferably a monoclonal antibody), the first binding partner being linked to a label (preferably an ECL label); (b) a second binding partner capable of specifically binding with the pathogen (preferably an antibody, more preferably a monoclonal antibody); and (c) a magnetic particle that is linked or capable of being linked to the second binding partner. Preferably, the kit further comprises at least one component selected from: (i) electrochemiluminescent co-reactant; (ii) pH buffer; and/or (iii) electrode for inducing electrochemiluminescence. Each reagent may be provided in wet form or in dry form (and rehydrated at time of use).

In one preferred embodiment, the second binding partner is an antibody of a certain species and/or class and the magnetic particle is coated with Protein A, Protein G, Protein L or a secondary antibody directed against antibodies of said certain species or class.

In another embodiment, the second binding partner is labeled with biotin and the magnetic particle is coated with avidin or streptavidin. Other useful capture reagents that, like biotin and streptavidin, are useful for capturing antibodies to solid phases are known in the art and may also be used.

Preferably, the first binding partner and/or the second binding partner are antibodies capable of specifically binding to the pathogen. More preferably, the first binding partner and/or the second binding partner are monoclonal antibodies capable of specifically binding to the pathogen.

Preferably, the kit further comprises at least one component selected from: buffers appropriate for carrying out the binding reaction (e.g., mixtures of pH buffering substances, detergents, salts, metal ions, cofactors, proteins, sugars, excipients, and the like), solutions appropriate for carrying out an ECL measurement, solutions appropriate for cleaning and/or conditioning an ECL measuring device, ECL labels, calibration solutions containing known concentrations of the pathogen(s) of interest, and calibration solutions for calibrating the response of an ECL measuring instrument.

Preferably, the kit further comprises an assay buffer, preferably an electrochemiluminescence assay buffer.

Preferably, the kit further comprises a pathogen positive control and/or a pathogen negative control.

Preferably, the kit further contains sample diluent, filters (for separating a test sample from a food or feces sample), and/or assay buffer.

According to one preferred embodiment, instead of specifically binding with the pathogen of interest, the first and/or second binding partner binds with a complex containing the pathogen of interest. For example, the pathogen of interest may first bind with a capture reagent forming a complex and the first binding partner and/or the second binding partner binds with the capture reagent thus linking the pathogen to the first binding partner and/or the second binding partner.

Preferably, the first binding partner is attached to a label, more preferably a luminescent label, even more preferably an electrochemiluminescent label, and even more preferably a metal containing electrochemiluminescent label, and/or the second binding partner is attached to a bead, more preferably attached to a magnetic microparticle having a diameter ranging from 1 to microns, even more preferably 1.5 to 3.0 microns.

Preferably, the components of the kit are packaged together in a common vessel or container or common package, optionally including instructions for performing a specific embodiment of the inventive methods. More preferably, one or more components are separated into one or more sub-containers within the common vessel, container or package.

The component(s) of the kit is or are typically kept separate by enclosing each in its own vial so as to eliminate cross-contamination or unintentional reaction prior to combination or use. Suitable containers for such a kit include, but are not limited to vials, bottles, boxes, tubes blister packs, cartridges, syringes, microtiter plates, ampules, and the like. Preferably, the kit is a package containing one or more separate containers containing different components of the kit.

One preferred embodiment relates to a kit for use in detecting or measuring Listeria comprising a first binding reagent capable of specifically binding to Listeria (preferably an antibody, more preferably a monoclonal antibody) and a second binding reagent capable of specifically binding to Listeria (preferably an antibody, more preferably a monoclonal antibody). More preferably, the kit contains a first binding reagent capable of specifically binding to Listeria, a second binding reagent capable of specifically binding to Listeria and a third binding reagent capable of binding to the second binding reagent. Even more preferably, the first binding reagent is linked to a label (preferably an electrochemiluminescent label), the second binding reagent is biotinylated and the third binding reagent is streptavidin coated beads, preferably streptavidin coated magnetic beads. According to a particularly preferred embodiment, the kit further comprises Listeria positive control and Listeria negative control. Preferably, the kit further comprises Listeria test sample diluent and/or sample preparation filter units. According to another preferred embodiment, the kit further contains Listeria enrichment broth (e.g., Becton Dickinson-Difco Listeria Enrichment Broth, modified (mLEB, catalog # 220530) or equivalent.

Another embodiment relates to a kit for use in detecting or measuring Salmonella comprising a first binding reagent capable of specifically binding to Salmonella (preferably an antibody, more preferably a monoclonal antibody) and a second binding reagent capable of specifically binding to Salmonella (preferably an antibody, more preferably a monoclonal antibody). More preferably, the first binding reagent is linked to a label (preferably an electrochemiluminescent label) and the second binding reagent is linked to beads, preferably magnetic beads. According to a particularly preferred embodiment, the kit further comprises Salmonella positive control and Salmonella negative control. Preferably, the kit further comprises Salmonella test sample diluent and/or sample preparation filter units. According to one preferred embodiment, the kit further contains phosphate buffered saline (e.g., phosphate buffer containing 0.5-200 mM phosphate and 0-150 mM sodium chloride with a pH range of 6.5-7.5), buffered peptone water, and/or Rappaport-Vassiliadis Soya (RVS) Peptone broth.

One preferred embodiment relates to a kit for use in detecting or measuring Campylobacter comprising a first binding reagent capable of specifically binding to Campylobacter (preferably an antibody, more preferably a monoclonal antibody) and a second binding reagent capable of specifically binding to Campylobacter (preferably an antibody, more preferably a monoclonal antibody). More preferably, the kit contains a first binding reagent capable of specifically binding to Campylobacter, a second binding reagent capable of specifically binding to Campylobacter and a third binding reagent capable of binding to the second binding reagent. Even more preferably, the first binding reagent is linked to a label (preferably an electrochemiluminescent label), the second binding reagent is biotinylated and the third binding reagent is streptavidin coated beads, preferably streptavidin coated magnetic beads. According to a particularly preferred embodiment, the kit further comprises Campylobacter positive control and Campylobacter negative control. Preferably, the kit further comprises Campylobacter test sample diluent and/or sample preparation filter units. According to another preferred embodiment, the kit further contains oxoid Bolton broth (CM983) or other Broth specific for growing Campylobacter and/or Oxoid Selective Supplements (SR183E) or other antimicrobial agents containing: cefoperazone 10 mg/500 ml, Trimethoprim 10 mg/500 ml, Vancomycin 10 mg/500 ml, Cycloheximide 25 mg/500 ml. Preferably, the kit further contains phosphate buffered saline.

One preferred embodiment relates to a kit for use in detecting or measuring E. coli O157 comprising a first binding reagent capable of specifically binding to E. coli O157 (preferably an antibody, more preferably a monoclonal antibody) and a second binding reagent capable of specifically binding to E. coli O157 (preferably an antibody, more preferably a monoclonal antibody). More preferably, the kit contains a first binding reagent capable of specifically binding to E. coli O157, a second binding reagent capable of specifically binding to E. coli O157 and a third binding reagent capable of binding to the second binding reagent, even more preferably, the first binding reagent is linked to a label (preferably an electrochemiluminescent label), the second binding reagent is biotinylated and the third binding reagent is streptavidin coated beads, preferably streptavidin coated magnetic beads. According to a particularly preferred embodiment, the kit further comprises E. coli O157 positive control and E. coli O157 negative control. Preferably, the kit further comprises Campylobacter test sample diluent and/or sample preparation filter units. Preferably, the kit further comprises a phosphate buffer (or Butterfeld's Solution or any phosphate buffer containing 0.5-200 mM phosphate and 0-150 mM sodium chloride with a pH range of 6.0-7.5) and/or a medium containing novobiocin.

Another embodiment of the invention relates to a reagent composition containing: (a) a first binding partner of the pathogen (preferably an antibody, more preferably a monoclonal antibody) and (b) a second binding partner of the pathogen (preferably an antibody, more preferably a monoclonal antibody). Preferably, the first binding partner is attached to a label, preferably a luminescent label, more preferably an electrochemiluminescent label and/or the second binding partner is attached to a bead, more preferably is attached to a magnetic microparticle having a diameter ranging from 1 to 5 microns. Preferably, the first and second binding partners are antibodies capable of specifically binding to the pathogen of interest.

Preferably, the reagent composition further comprises at least one component selected from: (i) luminescent label, preferably electrochemiluminescent label; (ii) electrochemiluminescent co-reactant; and/or (iii) electrode for inducing electrochemiluminescence.

Preferably, the reagent composition further comprises an assay buffer, preferably an electrochemiluminescence assay buffer.

Preferred embodiments of the invention also include reagent compositions containing the components of the inventive kits described above.

Another embodiment of the invention relates to an assay composition comprising any of the reagent compositions described above and a sample containing the pathogen of interest.

### EXAMPLES

The following examples are illustrative of some of the methods falling within the scope of the present invention. They are, of course, not to be considered in any way limitative of the invention. Numerous changes and modifications can be made with respect to the invention by one of ordinary skill in the art without undue experimentation.

The clinical utility of ORIGEN® electrochemiluminescent (ECL) immunoassays were evaluated in fecal samples. To evaluate the capacity of ORIGEN® technology, fecal samples were collected and tested for the presence of E. coli O157, Salmonella species, and Campylobacter species using IGEN International's PATHIGEN™ detection assays (PATHIGEN™ E. coli O157 test, Salmonella test, and Campylobacter test), which are currently used to detect food borne pathogens in food and water samples using conventional methods.

For pure bacterial cultures, the sensitivities of the PATHIGEN™ tests are in the range of 100-5000 bacteria. Direct detection of specific bacteria in a more complicated system, human feces, was performed for each pathogen tested. Thus, to assess the assays' performance clinically, fecal samples were subjected to routine culture techniques and the ORIGEN®-based tests. The assays took approximately 1.5 h without a need for enrichment of the samples. The example establish that the inventive methods for the detection of food borne pathogens in feces may provide a rapid clinical diagnosis of gastrointestinal infection. Surprisingly, the level of sensitivity was comparable to pathogens detected in food and water samples (e.g., spiking studies set forth in Example 2)

### Example 1

Human fecal samples from healthy patients and patients with known gastrointestinal disease (141 samples) were diluted to 10% using phosphate buffered saline and vortexed for approximately. 1 minute at a high speed. The vortexing action made a fecal slurry and broke-up any large particulates contained in the feces. The samples were then heat inactivated in a heat block at 80° C. for 15 minutes The fecal sample were then passed through a 200 um filter to remove the large particulates. A portion of the filtered sample (0.1 ml) was added to an ORIGEN® Analyzer test tube. A biotinylated antibody specific to the pathogen to be detected is added (0.025 ml) (anti-Campylobacter antibody, anti-Salmonella antibody, anti-E. Coli O157 antibody, which can be polyclonal or monoclonal antibodies and labeling ratios of 1-10 biotin per antibody) to the solution. In this example, the biotin used was ORIGEN® Biotin-LC-NHS and the antibodies were labeled with a 20 molar excess of biotin, purified by gel filtration chromatography, and stored at a concentration of 10-75 ug/ml in the PATHIGEN™ antibody dilutent containing: phosphate buffered saline, albumin (2% w/v), thesit (0.5% w/v), 2-Hydroxypyridine-N-oxide (0.1%), and N-methyl isothiazolone (0.1%), pH 6.8-7.8. A TAG-labeled antibody specific to the pathogen to be detected is added (0.025 ml) (anti-Campylobacter antibody, anti-Salmonella antibody, anti-E. coli O157 antibody, which can be polyclonal or monoclonal antibodies and labeling ratios of 1-3 TAG's per antibody) to the solution. In this example, the TAG used was ORI-TAGT™-NHS and the antibodies were labeled with a 4-8 molar excess of ORI-TAG™, purified by gel filtration chromatography, and stored at a concentration of 10-75 ug/ml in the PATHIGEN antibody dilutent containing: phosphate buffered saline, albumin (2% w/v), thesit (0.5% w/v), 2-Hydroxypyridine-N-oxide (0.1%), and N-methyl isothiazolone (0.1%). A streptavidin-coated paramagnetic microparticle solution (0.025 ml) containing DYNAL®-streptavidin M-280 microparticles stored at a concentration of 0.5-1 mg/ml, HEPES buffer (1.3% w/v), sucrose (2%), thesit (0.1% w/v), albumin (0.1%), 2-Hydroxypyridine-N-oxide (0.1%), and N-methyl isothiazolone (0.1%) is added to the solution. A buffer (0.5 ml) is added (the buffer was phosphate buffered saline for the Salmonella and E. Coli O157 tests) and PATHIGEN™ Sample Buffer containing 20 mM phosphate buffer, pH 5.5, thesit (2.7% w/v), albumin (0.5%), 2-Hydroxypyridine-N-oxide (0.1%), and N-methyl isothiazolone (0.1%) is added to the solution The sample was incubated for 60 minutes to allow the antigen to interact with the antibodies and the magnetic beads. The sample was then added to an ORIGEN® Analyzer for electrochemiluminescent stimulation. Tables 2, 3, and 4 (below) show the rapid screening of several samples from healthy and diseased patients. Campylobacter and Salmonella were detected by both culture and ORIGEN® analysis.

TABLE 1
Campylobacter Test - Screening fecal samples for
the presence of Campylobacter species

| | | ECL Signal | | |
|---|---|---|---|---|
| | Number of | Mean | Signal/ | |
| Sample | samples | Standard | Back- | Culture |
| Type | tested | deviation | ground | Results |
| Healthy Individuals | 108 | 14806 +/− | 1.0 +/− | All |
| | | 9905 | 0.9 | negative |
| Diseased Individuals | 30 | 31334 +/− | 2.3 +/− | All |
| (Individuals with known | | 25469 | 1.9 | negative |
| gastrointestinal disease) | | | | |
| Campylobacter positive | 1 | 306962 | 37.9 | Positive |
| sample | | | | |
| Campylobacter positive | 1 | 107660 | 13.3 | Positive |
| sample | | | | |
| Campylobacter positive | 1 | 21906 | 2.7 | Positive |
| sample | | | | |
| Positive Control | 5 | 566197 +/− | 41.2 +/− | NA |
| | | 125451 | 20.6 | |
| Negative Control | 5 | 16824 +/− | 1.0 | NA |
| | | 8964 | | |

NA = not applicable

Table 1 shows the ECL signal readout and signal-to-background calculations. For the detection step, 0.1 mL of sample was used for the detection portion of the assay. Samples from healthy individuals did not react in the assay. Stools from patients with known gastrointestinal disease (Campylobacter was not isolated from these stools) showed a higher, however, insignificant signal/background ratio over the negative controls. Three of three stools that were positive for Campylobacter by culture methods were also positive using the PATHIGEN™ Campylobacter test.

TABLE 2
Salmonella test - Screening fecal samples for the
presence of Salmonella Species.

| | | ECL Signal | | |
|---|---|---|---|---|
| | Number of | Mean | Signal/ | |
| Sample | samples | Standard | Back- | Culture |
| Type | tested | deviation | ground | Results |
| Healthy Individuals | 108 | 6345 +/− | 0.8 +/− | All |
| | | 1488 | 0.2 | negative |
| Diseased Individuals | 30 | 7200 +/− | 1.3 +/− | All |
| (Individuals with known | | 5180 | 0.9 | negative |
| gastrointestinal disease) | | | | |
| Salmonella positive | 1 | 209807 | 36.9 | Positive |
| sample | | | | |
| Salmonella positive | 1 | 299515 | 52.5 | Positive |
| sample | | | | |
| Positive Control | 5 | 104079 +/− | 16.2 +/− | NA |
| | | 5988 | 3.8 | |
| Negative Control | 5 | 6715 +/− | 1.0 | NA |
| | | 1600 | | |

Table 2 shows the ECL signal readout and signal-to-background calculations for several Salmonella assays. For detection, 0.1 mL was used for the detection portion of the assay. Samples from healthy individuals did not react in the assay. Stools from patients with known gastrointestinal disease (Salmonella was not isolated from these stools) showed a higher, however, insignificant signal/background ratio over the negative controls. Two stools that were positive for Salmonella by culture methods were also positive using the PATHIGEN™ Salmonella test

TABLE 3
E. coli O157 test - Screening fecal samples
for the presence of E. coil O157.

| Sample | Number of | ECL Signal Mean | Signal/ |
|---|---|---|---|
| Type | samples | Standard deviation | Background |
| Healthy Individuals | 108 | 4076.1 +/− | 0.2 +/− |
| | | 2001.2 | 0.1 |
| Diseased Individuals | 30 | 3852.1 +/− | 0.3 +/− |
| (Individuals with known | | 1990.0 | 0.2 |
| gastrointestinal disease) | | | |
| Positive Control | 5 | 135366.6 +/− | 91 +/− |
| | | 28947.9 | 0.7 |
| Negative Control | 5 | 15071.8 +/− | 1.0 |
| | | 2398.1 | |

Table 3 shows the ECL signal readout and signal-to-background calculations. For detection, 0.1 mL of sample was used for the detection portion of the assay. Samples from healthy individuals did not react in the assay. Stools from patients with known gastrointestinal disease (E. coli O157 was not isolated from these stools) showed a higher, however, insignificant signal/background ratio over the negative controls.

### Example 2

Human fecal samples were thawed and divided into 1-gram quantities in 9 mL of sterile phosphate buffered saline. Samples were vortexed until they became fecal slurries (˜1 minute). To test for the sensitivity of the PATHIGEN™ Tests in the fecal matrix, samples were spiked with Campylohacter jejuni, Salmonella Typhimurium, and E. coli O157 bacteria at various concentrations, 0.1 mL of the diluted bacterial solution was added to the respective fecal samples and vortexed. Samples were then heat inactivated using a heat block at 80° C. for 15 min and filtered through a 200 um filter to remove the large particulates. A portion of the filtered sample (0.1 ml) was added to an ORIGEN® Analyzer test tube. A biotinylated antibody specific to the pathogen to be detected is added (0.025 ml) (anti-Campylobacter antibody, anti-Salmonella antibody, anti-E. coli O157 antibody, which can be polyclonal or monoclonal antibodies and labeling ratios of 1-10 biotin per antibody) to the solution. In this example, the biotin used was ORIGEN® Biotin-LC-NHS and the antibodies were labeled with a 20 molar excess of biotin, purified by gel filtration chromatography, and stored at a concentration of 10-75 ug/ml in the PATHIGEN™ antibody dilutent containing: phosphate buffered saline, albumin (2% w/v), thesit (0.5% w/v), 2-Hydroxypyridine-N-oxide (0.1%), and N-methyl isothiazolone (0.1%), pH 6.8-7.8. A TAG-labeled antibody specific to the pathogen to be detected is added (0.025 ml) (anti-Campylobacter antibody, anti-Salmonella antibody, anti-E. coli O157 antibody, which can be polyclonal or monoclonal antibodies and labeling ratios of 1-3 TAG's per antibody) to the solution. In this example, the TAG used was ORI-TAG™-NHS and the antibodies were labeled with a 4-8 molar excess of ORI-TAG™, purified by gel filtration chromatography, and stored at a concentration of 10-75 ug/ml in the PATHIGEN™ antibody dilutent containing: phosphate buffered saline, albumin (2% w/v), thesit (0.5% w/v), 2-Hydroxypyridine-N-oxide (0.1%), and N-methyl isothiazolone (0.1%). A streptavidin-coated paramagnetic microparticle solution (0.025 ml) containing DYNAL®-streptavidin M-280 microparticles stored at a concentration of 0.5-1 mg/ml, HEPES buffer (1.3% w/v), sucrose (2%), thesit (0.1% w/v), albumin (0.1%), 2-Hydroxypyridine-N-oxide (0.1%), and N-methyl isothiazolone (0.1%) is added to the solution. A buffer (0.5 ml) is then added. The buffer was phosphate buffered saline for the Salmonella and E. coli O157 tests and PATHIGEN™ Sample Buffer (containing 20 mM phosphate buffer, pH 5.5, thesit (2.7% w/v), albumin (0.5%), 2-Hydroxypyridine-N-oxide (0.1%), and N-methyl isothiazolone (0.1%)) for the Campylobactor test. The sample was incubated for 60 minutes to allow the antigen to interact with the antibodies and the magnetic beads. The sample was then added to an ORIGEN® Analyzer for electrochemiluminescent stimulation.

Tables 4, 5, and 6 show the detection of Campylobacter, Salmonella, and E. coli O157 that were spiked into fecal samples to determine the sensitivity in the fecal matrix.

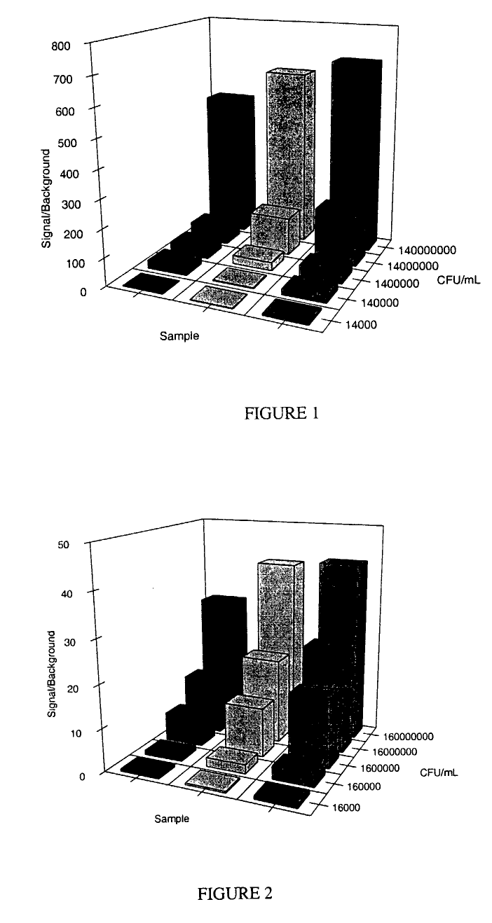

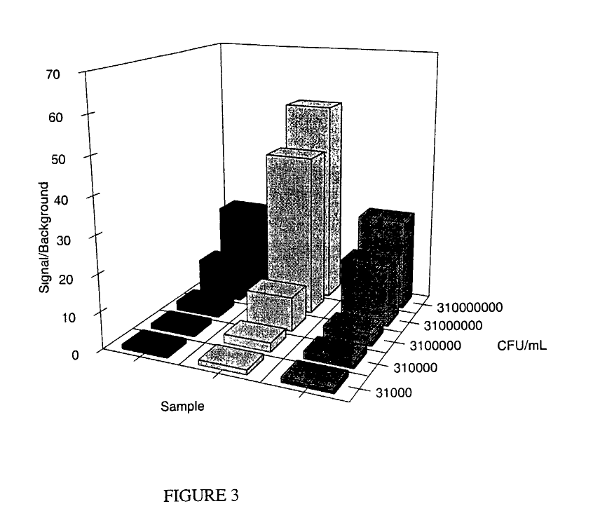

Moreover, data demonstrate the surprising and unexpected results achieved using the invention FIG. 1 is a graphical representation of the data in Table 4; FIG. 2 is a graphical representation of the data in Table 5; and FIG. 3 is a graphical representation of the data in Table 6.

TABLE 4
PATHIGEN ™ Campylobacter Test Sensitivity in the Matrix analysis
C. jejuni

| Cell count | Sample 1 | | Sample 2 | | Sample 3 | |
|---|---|---|---|---|---|---|
| CFU/mL | ECL | S/B | ECL | S/B | ECL | S/B |
| 1.4 × 10⁸ | 4146396 | 512.2 | 5081710 | 627.8 | 5519201 | 681.8 |
| CFU/mL | | | | | | |
| 1.4 × 10⁷ | 483191 | 59.7 | 1086512 | 134.2 | 1520902 | 187.9 |
| CFU/mL | | | | | | |
| 1.4 × 10⁶ | 325078 | 40.2 | 244054 | 30.1 | 331610 | 40.9 |
| CFU/mL | | | | | | |
| 1.4 × 10⁵ | 248148 | 30.7 | 28633 | 3.5 | 173725 | 21.5 |
| CFU/mL | | | | | | |
| 1.4 × 10⁴ | 20446 | 2.5 | 19323 | 2.4 | 54697 | 6.8 |
| CFU/mL | | | | | | |

| | | |
|---|---|---|
| Positive control | 335228 | 41.4 |
| Negative control | 8095 | 1.0 |

The data shown in Table 4 indicates a detection limit of around 1×10⁵ CFU/gram of feces for the Campylobacter test.

TABLE 5
PATHIGEN ™ E. coli O157 Test Sensitivity in the Matrix analysis

| | E. coli O157 | | | | | |
|---|---|---|---|---|---|---|
| Cell count | Sample 1 | | Sample 2 | | Sample 3 | |
| CFU/mL | ECL | S/B | ECL | S/B | ECL | S/B |
| 1.6 × 10⁸ | 231435 | 30.4 | 309903 | 40.7 | 317534 | 41.7 |
| CFU/mL | | | | | | |
| 1.6 × 10⁷ | 99192 | 13.0 | 151636 | 19.9 | 186053 | 24.4 |
| CFU/mL | | | | | | |
| 1.6 × 10⁶ | 51551 | 6.8 | 87588 | 11.5 | 123230 | 16.2 |
| CFU/mL | | | | | | |
| 1.6 × 10⁵ | 8380 | 1.1 | 16075 | 2.1 | 23473 | 3.1 |
| CFU/mL | | | | | | |
| 1.6 × 10⁴ | 5171 | 0.7 | 2771 | 0.4 | 6888 | 0.9 |
| CFU/mL | | | | | | |

| | | |
|---|---|---|
| Positive control | 159072 | 20.9 |
| Negative control | 7615 | 1.0 |

The data shown in Table 5 indicates a detection limit of around 1×10⁵ CFU/gram of feces for the E. coli O157 test.

TABLE 6
PATHIGEN ™ Salmonella Test Sensitivity in the Matrix analysis

| | S. typhimurium | | | | | |
|---|---|---|---|---|---|---|
| Cell count | Sample 1 | | Sample 2 | | Sample 3 | |
| CFU/mL | ECL | S/B | ECL | S/B | ECL | S/B |
| 3.1 × 10⁸ | 125662 | 22.1 | 312759 | 55.0 | 142071 | 25.0 |
| CFU/mL | | | | | | |
| 3.1 × 10⁷ | 57691.5 | 10.1 | 247609 | 43.6 | 94736 | 16.7 |
| CFU/mL | | | | | | |
| 3.1 × 10⁶ | 14728 | 2.6 | 52797 | 9.3 | 18756 | 3.3 |
| CFU/mL | | | | | | |
| 3.1 × 10⁵ | 8230 | 1.4 | 13671 | 2.4 | 11157 | 2.0 |
| CFU/mL | | | | | | |
| 3.1 × 10⁴ | 7372 | 1.3 | 7321 | 1.3 | 8280 | 1.5 |
| CFU/mL | | | | | | |

| | | |
|---|---|---|
| Positive control | 120274 | 21.2 |
| Negative control | 5685 | 1.0 |

The data shown in Table 6 indicates a detection limit of around 5×10⁵ CFU/gram of feces with the Salmonella test.

Thus, the above-described examples confirm the surprising and unexpected results achieved using the present invention. More specifically, the ability to rapidly and accurately detect low levels of specific pathogens without the time-consuming and cumbersome steps of conventional methods.

The terms and expressions which have been employed are used as terms of description and not of limitations, and there is no intention in the use of such terms or expressions of excluding any equivalents of the features shown and described as portions thereof, its being recognized that various modifications are possible within the scope of the invention.

1. A method for detecting a bacterial pathogen in a sample comprising:
(a) diluting said sample to form a diluted sample;
(b) forming a slurried sample from said diluted sample;
(c) inactivating said slurried sample to form an inactivated slurried sample;
(d) forming a composition containing:
(i) said inactivated slurried sample;
(ii) an assay-performance-substance linked to an electrochemiluminescent compound and containing at least one component selected from the group consisting of:
(1) added bacterial pathogen or added analogue of said bacterial pathogen;
(2) a binding partner of said bacterial pathogen or a binding partner of said analogue; and
(3) a component capable of binding with (1) or (2); and
(iii) a plurality of particles capable of specifically binding with said bacterial pathogen, said assay-performance-substance or combinations thereof;
(e) incubating said composition to form a complex containing said particles and said electrochemiluminescent compound;
(f) inducing electrochemiluminescence from said electrochemiluminescent compound; and
(g) detecting emitted luminescence.

2. A method for detecting a bacterial pathogen in a sample comprising:
(a) diluting said sample to form a diluted sample;
(b) forming a slurried sample from said diluted sample;
(c) inactivating said slurried sample to form an inactivated slurried sample;
(d) removing solids from said inactivated slurried sample to form a supernatant sample;
(e) forming a composition containing:
(i) said supernatant sample;
(ii) an assay-performance-substance linked to an electrochemiluminescent compound and containing at least one component selected from the group consisting of:
(1) added bacterial pathogen or added analogue of said bacterial pathogen.
(2) a binding partner of said bacterial pathogen or a binding partner of said analogue; and
(3) a component capable of binding with (1) or (2); and
(iii) a plurality of inanimate particles capable of specifically binding with said bacterial pathogen and/or said assay-performance-substance;
(f) incubating said composition to form a complex containing said inanimate particles and said electrochemiluminescent compound;
(g) collecting said inanimate particles in a zone where electrochemiluminescence can be induced to occur;
(h) inducing electrochemiluminescence; and
(i) detecting emitted luminescence,
wherein said sample is selected from the group consisting of a food sample, a fecal sample, or water sample.

3. The method of claim 1, wherein said method consists essentially of steps (a) through (g).

4. The method of claim 2, wherein said method consists essentially of steps (a) through (i).

5. The method of claim 1, wherein said method consists of steps (a) through (g).

6. The method of claim 2, wherein said method consists of steps (a) through (i).

7. The method of claim 1, wherein said pathogen is one or more specific pathogens.

8. The method of claim 1, wherein said bacterial pathogen is selected from the group consisting of E. coli, Salmonella species, and Campylobacter species.

9. The method of claim 1, wherein said method provides sensitivities as low as 100 to 50000 cells when detected without enrichment of said sample.

10. The method of claim 1, wherein said method provides sensitivities as low as 100 to 500 cells when detected without enrichment of said sample.

11. The method of claim 1, wherein said bacterial pathogen is E. coli O157 and said method provides a detection limit as low as 1×10⁵ CFU/gram of feces without enrichment of said sample.

12. The method of claim 1, wherein said bacterial pathogen is Salmonella and said method provides a detection limit as low as 5×10⁵ CFU/gram of feces without enrichment of said sample.

13. The method of claim 1, wherein said bacterial pathogen is Campylobacter and said method provides a detection limit as low as 1×10⁴ CFU/gram of feces without enrichment of said sample.

14. The method of claim 1, wherein said sample is an unenriched sample.

15. The method of claim 1, wherein said sample is a direct fecal sample.

16. The method of claim 1, wherein said sample is a fecal sample and said fecal sample is diluted to at least 10% weight/volume in phosphate buffered saline.

17. The method of claim 1, wherein said slurried sample is formed by vortexing said diluted sample.

18. The method of claim 1, wherein said inactivating comprises heat inactivation

19. The method of claim 2, wherein said solids are removed by filtering.

20. The method of claim 1, wherein said method is performed in less than 3 hours.

21. The method of claim 1, wherein said pathogen is Listeria.

22. A method for detecting a bacterial pathogen in a fecal sample comprising:
(a) forming a composition containing said fecal sample and an assay-performance-substance linked to an electrochemiluminescent compound and containing at least one component selected from the group consisting of.
(1) added bacterial pathogen or added analogue of said bacterial pathogen;
(2) a binding partner of said bacterial pathogen or a binding partner of said analogue; and
(3) a component capable of binding with (1) or (2); and
(b) inducing electrochemiluminescence from said electrochemiluminescent compound; and
(c) detecting emitted luminescence

23. The method of claim 22, wherein said method does not include a sample enrichment step.

24. An electrochemiluminescence based immunoassay for detecting a pathogen in a sample wherein said assay method does not comprise an enrichment step.
